(19) United States
(12) Patent Application Publication
(10) Pub. No.: US 2005/0009161 A1
(43) Pub. Date: Jan. 13, 2005

(54) Enhancement of in vitro culture or vaccine production using electromagnetic energy treatment

(76) Inventor: Jackson Streeter, Reno, NV (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/700,355

(22) Filed: Nov. 3, 2003

Related U.S. Application Data
(60) Provisional application No. 60/423,643, filed on Nov. 1, 2002.
(60) Provisional application No. 60/488,490, filed on Jul. 17, 2003.

(51) Int. Cl. C12N 13/00; C12M 3/00

(52) U.S. Cl. 435/173.8; 435/289.1

(57) ABSTRACT

Disclosed are apparatus and methods for enhancing or improving cell cultures, including cell cultures for the production of monoclonal antibodies, using electromagnetic energy treatment, primarily using light in the near infrared to visible region of the spectrum. The delivery of light energy to a culture, in accordance with preferred embodiments, enhances or improves the cell culture such as by providing for enhanced and accelerated formation of important biological macromolecules, including, but not limited to, antibodies, proteins, collagen, and polysaccharides, and also providing for accelerated cellular replication and an enhancement or prolongation of the life of cells so treated.

### RELATED APPLICATION DATA

This application claims priority under 35 U.S.C. § 119(e) to U.S. Provisional Patent Application Nos. 60/423,643 filed Nov. 1, 2002 and 60/488,490 filed Jul. 17, 2003, the disclosures of which are hereby incorporated by reference in their entireties.

### FIELD OF THE INVENTION

The present invention relates in general to methods for enhancing or improving cell cultures, including cell cultures for the production of monoclonal antibodies, bacteria, or other useful materials, using electromagnetic energy treatment, primarily light in the near infrared to visible region of the spectrum.

### BACKGROUND OF THE INVENTION

In vitro cell cultures are used in a variety of contexts, including in biotechnology. Important uses of cell culture include the culturing of bacteria or hybridomas for the large-scale production of macromolecules such as antibodies or other proteins that are useful as biotechnological drugs, the culturing of bacteria useful for vaccines, and culturing of animal cells containing viruses useful for biotechnology or vaccines. Because obtaining a drug agent or vaccine material via cell culture can be expensive, especially as compared to many synthetic methods used for small molecule pharmaceuticals, there is a need for a method to increase the yield and efficacy of such cell cultures.

### SUMMARY OF THE INVENTION

The electromagnetic energy treatment methods, also called low level light treatment methods, for enhancing or improving cell cultures is based in part on the discovery that light energy applied to a culture enhances or improves the cell culture such as by providing for enhanced and accelerated formation of important biological macromolecules, including, but not limited to, antibodies, proteins, collagen, and polysaccharides, and also providing for accelerated cellular replication and an enhancement or prolongation of the life of cells so treated. Methods disclosed in accordance with the preferred embodiments herein may be used to accelerate the production of vaccines and/or other important products containing biological materials.

In accordance with one embodiment there are provided methods directed toward enhancing or improving the performance of a cell culture. The methods include delivering an effective amount of electromagnetic (light) energy having a wavelength in the visible to near-infrared wavelength range to cells in a culture, wherein delivering the effective amount of light energy includes delivering a predetermined power density of light energy to the cells in culture and wherein the delivering the light results in the enhancement or improvement of the cell culture.

In one embodiment the predetermined power density is a power density of at least about 0.01 mW/cm². The predetermined power density is typically selected from the range of about 0.01 mW/cm² to about 100 mW/cm², including from about 0.01 mW/cm² to about 15 mW/cm² and from about 2 mW/cm² to about 50 mW/cm².

In preferred embodiments, the methods encompass using light energy having a wavelength of about 630 nm to about 904 nm, and in one embodiment the light energy has a wavelength of about 780 nm to about 840 nm. The light energy is preferably from a coherent source (i.e. a laser), but light from non-coherent sources may also be used.

In a related embodiment, there is provided a cell culture apparatus including a reservior for holding the cells and culture medium, an ambient conditions control system which controls variables such as the temperature of the culture, CO₂ levels, and other conditions necessary for cell growth and maintenance, and a light delivery device comprising at least one light source adapted to deliver electromagnetic energy to the cell culture, wherein light delivered by the light delivery device results in the enhancement or improvement of the cell culture.

### DETAILED DESCRIPTION OF THE PREFERRED EMBODIMENTS

The term “cell” as used herein is a broad term used in its ordinary sense and includes animal cells such as human or mammalian cells, hybridomas, and single-celled organisms such as bacteria. A “cell culture” includes one or more cells in a medium that provides for the growth of the one or more cells. The cell culture may be of any type, including small-scale cultures such as are performed in small dishes or plates as are commonly used in research laboratories as well as large-scale cultures performed in large vessels or vats as are commonly used in the pharmaceutical and biotech industries for cultures to produce and harvest biological macromolecules on a pilot plant or commercial scale.

Terms such as “enhancement” or “enhance” as used with regard to cells or cell culture refers to an improvement of properties of the culture or cells as compared to a culture or cells that do not receive treatment, such improved properties including enhanced and accelerated formation of important biological macromolecules, including, but not limited to, antibodies, proteins, collagen, and polysaccharides by the cell, accelerated cellular replication, and prolongation of the life the cell or cells.

The low level light treatment methods may be practiced using, for example, a low level laser therapy apparatus such as that shown and described in U.S. Pat. No. 6,214,035, U.S. Pat. No. 6,267,780, U.S. Pat. No. 6,273,905 and U.S. Pat. No. 6,290,714, which are all herein incorporated by reference together with references contained therein.

Light delivery devices other than those noted above may also be used. Characteristics of preferred light delivery devices include the presence of one or more light energy sources. The one or more sources may be disposed on a plate or panel that can be moved or positioned as desired or they may be fixed in place. In one embodiment, one or more sources are fixed to one or more inside surfaces of a vessel used for cell culture. Alternatively, the sources may be on a support that is removable from the vessel. In any case, the sources of the device should be positioned so as to irradiate the cells in the culture.

Preferred sources are generally of the coherent variety (i.e. lasers), however non-coherent sources may also be used, or a combination of coherent and non-coherent sources. The one or more sources are capable of emitting light energy having a wavelength in the visible to near-infrared wavelength range, preferably about 630 nm to about 904 nm, including about 780 nm to about 840 nm, including about 790, 800, 810, 820, and 830 nm. In one embodiment, the source is a continuously emitting GaAlAs laser diode having a wavelength of about 830 nm. In another embodiment, a laser source is used having a wavelength of about 808 nm. In preferred embodiments, the light produced is substantially monochromatic (i.e. one wavelength or a very narrow band of wavelengths).

In preferred embodiments of light delivery devices, there is a power supply operatively coupled to the light source or sources, and a programmable controller operatively coupled to the light source or sources and to the power supply. The programmable controller is preferably configured to select a predetermined power density of the light energy to be delivered to the cell culture and/or other properties such as pulsing, time of treatment, frequency of treatment, and the like.

During the treatment, the light energy may be continuously provided, or it may be pulsed. If the light is pulsed, the pulses are preferably at least about 10 ns long and occur at a frequency of up to about 100 Hz. Time between pulses may be longer or shorter than the time of the pulse, and can vary, for example, from a few nanoseconds to several seconds or minutes. Continuous wave light may also be used. The pulsing, time between pulses, and the length of pulses are preferably set and controlled using the programmable controller.

In accordance with a preferred embodiment, the predetermined power density is about 0.01 mW/cm² to about 100 mW/cm², including about 0.05, 0.1, 0.5, 1, 5, 10, 15, 20, 30, 40, 50, 60, 70, 80, and 90 mW/cm². In one embodiment, power densities of about 20 mW/cm² to about 50 mW/cm² are used. To achieve the preferred power densities, preferred light energy sources, or light energy sources in combination, are capable of emitting light energy having a total power output of about 0.1 mW to about 500 mW, including about 0.5, 1, 5, 10, 20, 30, 50, 75, 100, 150, 200, 250, 300, and 400 mW, but may also be up to about 1000 mW.

The precise power density selected for treating the culture depends on a number of factors, including the specific wavelength of light selected, the type of cells, the particular macromolecule(s) or cell behavior desired, the medium, and the like. For example, when the cell culture is in a container having a large volume, one may take into account attenuation of the energy of the light as it travels through the culture medium to reach cells at a greater distance from the source. If, however, the culture is stirred or similarly manipulated, the need to account for attenuation may be obviated in that all cells in the culture will receive substantially equal energy. Similarly, it should be understood that the power density of light energy to be delivered to the culture may be adjusted to be combined with any other therapeutic agent or agents to achieve a desired biological effect. The selected power density will again depend on a number of factors, including the specific light energy wavelength chosen, the individual additional therapeutic agent or agents chosen, and the cell line used.

In preferred embodiments, treatment comprises one or more treatment periods. A treatment period may last for anywhere from a few seconds to several hours, days or weeks. If there is more than one treatment period, the time between treatment periods can be from one or more hours to several days. In one embodiment, the treatment is divided into at least ten periods, each period lasting about one hour during which the light is delivered in a series of pulses, with a time of at least about six hours passing between the treatment periods.

Light delivery devices and sources having power capacities, wavelengths and other properties outside of the limits set forth above may also be used in accordance with the methods disclosed herein.

Preferred methods for the treatment of cells in culture involve delivering light energy having a wavelength in the visible to near-infrared wavelength range to cells in the culture, wherein delivering the light results in the enhancement or improvement of the cell culture or properties thereof. Delivering the light energy includes selecting a power density of the light energy, preferably at least about 0.01 mW/cm². Preferred embodiments include or further include one or more of the following: the light energy is delivered as a series of pulses; the wavelength of the light is about 780 nm to about 840 nm; the light source is a coherent source; and the treatment is conducted in at least two treatment periods.

In one embodiment, preferred methods are performed using a cell culture apparatus adapted for performing the methods. The cell culture apparatus includes a reservior, plate, dish, vessel, support, or other apparatus for holding or containing the cells and culture medium, an ambient conditions control system which controls variables such as the temperature of the culture, CO₂ and/or other gas levels, and other conditions for cell growth and maintenance. The cell culture apparatus also comprises a light delivery device, as disclosed hereinabove in accordance with preferred embodiments, comprising at least one light source adapted to deliver electromagnetic energy to the cell culture, wherein light delivered by the light delivery device results in the enhancement or improvement of the cell culture. By enhancing or improving the cell culture, the production of the products derived from the cell culture is also enhanced or accelerated, such products being useful as drugs, vaccines, and the like. Many types of cell culture apparatus are well known in the art, including, but not limited to, large and small scale incubators, and large and small scale bioreactors. Such apparatus can be readily adapted to include a light delivery device or source in accordance with the disclosure herein.

The explanations and illustrations presented herein are intended to acquaint others skilled in the art with the invention, its principles, and its practical application. Those skilled in the art may adapt and apply the invention in its numerous forms, as may be best suited to the requirements of a particular use. Accordingly, the specific embodiments of the present invention as set forth are not intended as being exhaustive or limiting of the invention.

1. A method for the treatment of cells in a cell culture, comprising delivering an effective amount of electromagnetic energy having a wavelength in the visible to near-infrared wavelength range to cells in a culture, wherein delivering the effective amount of light energy includes delivering light having a power density of at least about 0.01 mW/cm² to the cells in culture and wherein the delivering the light results in the enhancement or improvement of the cell culture.

2. A method according to claim 1 wherein the power density is about 0.01 mW/cm² to about 100 mW/cm².

3. A method according to claim 2 wherein the power density is about 0.01 mW/cm² to about 15 mW/cm².

4. A method according to claim 1 wherein the light energy has a wavelength of about 630 nm to about 904 nm.

5. A method according to claim 4 wherein the light energy has a wavelength of about 780 nm to about 840 nm.

6. A method according to claim 1 wherein delivering comprises placing a light source above a top surface of a container holding a cell culture.

7. A method according to claim 1 wherein delivering comprises delivering a series of pulses of light.

8. A method according to claim 1 wherein the treatment is broken into at least two treatment periods.

9. A method according to claim 1, wherein the treatment proceeds for a period of about 30 seconds to about 2 hours.

10. A cell culture apparatus, comprising
a reservior for holding the cells and culture medium;
an ambient conditions control system for controlling variables such as the temperature of the culture, CO₂ levels, and other conditions necessary for cell growth and maintenance; and
a light delivery device comprising at least one light source adapted to deliver electromagnetic energy to the cell culture, wherein light delivered by the light delivery device results in the enhancement or improvement of the cell culture.

11. A method for accelerating the production of a vaccine, comprising delivering an effective amount of electromagnetic energy having a wavelength in the visible to near-infrared wavelength range to cells in a culture, wherein delivering the effective amount of light energy includes delivering light having a power density of at least about 0.01 mW/cm² to the cells in culture; wherein the delivering the light results in the enhancement or improvement of the cell culture; and wherein the cultured cells or products thereof are useful in a vaccine.

12. A method according to claim 11 wherein the power density is about 0.01 mW/cm² to about 100 mW/cm².

13. A method according to claim 12 wherein the power density is about 0.01 mW/cm² to about 15 mW/cm².

14. A method according to claim 11 wherein the light energy has a wavelength of about 630 nm to about 904 nm.

15. A method according to claim 14 wherein the light energy has a wavelength of about 780 nm to about 840 nm.

16. A method according to claim 11 wherein delivering comprises placing a light source above a top surface of a container holding a cell culture.

17. A method according to claim 11 wherein delivering comprises delivering a series of pulses of light.

18. A method according to claim 11 wherein the treatment is broken into at least two treatment periods.

19. A method according to claim 11, wherein the treatment proceeds for a period of about 30 seconds to about 2 hours.
